(19) United States
(12) Patent Application Publication
(10) Pub. No.: US 2023/0331905 A1
(43) Pub. Date: Oct. 19, 2023

(54) BIOBASED CURING AGENTS FOR EPOXY RESINS

(71) Applicant: THE GOVERNORS OF THE UNIVERSITY OF ALBERTA, Edmonton (CA)

(72) Inventors: Jonathan CURTIS, Edmonton (CA); Vinay PATEL, Edmonton (CA)

(73) Assignee: THE GOVERNORS OF THE UNIVERSITY OF ALBERTA, Edmonton, AB (CA)

(21) Appl. No.: 18/044,556

(22) PCT Filed: Sep. 21, 2021
(86) PCT No.: PCT/CA2021/051247

§ 371 Date: Mar. 8, 2023

Related U.S. Application Data
(60) Provisional application No. 63/075,940, filed on Sep. 9, 2020.

(51) Int. Cl. C08G 59/42; C07C 69/704; C07C 67/08

(52) U.S. Cl. CPC C08G 59/4207; C07C 69/704; C07C 67/08

(57) ABSTRACT

Curing agents for epoxy monomers are prepared from the partial esterification of citric acid with certain alkyl or alkenyl alcohols. These curing agents, which contain mixtures of unreacted citric acid and monoalkyl or monoalkenyl citrate, and which are substantially free of trialkyl or trialkenyl citrate, are used without co-solvents for preparing biobased resins from plant oil-derived epoxides. The resulting biobased resins can in turn be used to prepare thermo set materials and biocomposites.

### TECHNICAL FIELD

The invention relates to preparing biobased curing agents from partial esters of citric acid, and to methods for preparing bioresins containing such partial esters in the presence of epoxy monomers. Related biobased curing agents and bioresins are also provided.

### DESCRIPTION OF THE RELATED ART

Due to environmental concerns, nonrenewable based materials are being replaced with biobased materials. About 10% of the total fossil-based feedstock is utilized to make various chemicals and materials. The majority of commercial epoxy resins and curing agents are derived from nonrenewable resources, and attempts are being made to show that a variety of renewable sources can be utilized to make similar materials with high biocontent.

Epoxy monomers can be derived from renewable resources like unsaturated plant-based triacylglyceride oils, but also possibly from waste animal fat. Many plant-oil-epoxy technologies known in the prior art require co-solvents in the manufacture of thermoset materials because many curing agents are immiscible with the plant-oil-epoxides, especially at ambient temperatures.

Citric acid (C₆H₈O₇) is a biobased, multifunctional compound that is readily available commercially, being produced in industrial quantities for the food industry through fermentation. Citric acid has been shown to be useful as a curing agent as it contains three carboxylic acid groups which are available to participate in cross-linking reactions as well as one hydroxyl group. However, a major limitation to using citric acid is that it is insoluble in plant-oil-epoxides and has a relatively high melting point. One potential solution involves the production of a pre-polymer by controlled heating of an epoxide/citric acid/solvent mixture. However, this is not a convenient solution because it results in toxic solvent vapours which are undesirable in industrial applications, and importantly, results in a prepolymer with a limited pot-life.

Epoxy monomers are vital building blocks for making thermoset polymers. Such thermoset polymers can be used in adhesives and sealants, as resins in making composite materials including biocomposites which incorporate natural fibres, in coatings, and in many other applications. The properties of these thermoset materials are highly dependent on the structures of the epoxy monomers and the curing agents.

### BRIEF SUMMARY

Despite citric acid's unique features, it is not suitable to be used with plant oil epoxides because it is immiscible with them. This invention is based, in part, on the identification of a solvent free synthetic route for making citric acid derivatives compatible with plant oil epoxides to manufacture bioresins.

Aspects of the invention comprise methods of preparing multifunctional partial-esters of citric acid for use as an epoxy curing agent for use in the manufacture of biobased thermosets, preferably via solvent-free processes. The invention described here includes the synthesis of citric acid derivatives which retain 2 or more acid groups, but which are substantially or fully miscible with plant-oil-epoxides. This eliminates or minimizes the need for solvent use in the preparation of the resin, and so may substantially eliminate solvent from the thermoset material.

In some embodiments, citric acid is partially-esterified in a controlled manner via a solvent-free and catalyst-free partial esterification procedure with an alcohol, preferably shortchain alcohols, which are also preferably biobased (for example, ethanol, propanol, butanol). This synthesis may be controlled in a manner that the entire reaction product, which preferably contains a mixture of free citric acid and mono-alkyl citrate, becomes a single liquid phase and which is fully miscible with plant-oil-epoxides. The reaction product may also comprise small amounts of dialkyl or trialkyl citrate, depending on reaction conditions.

Accordingly, in an embodiment, the present invention provides a method of preparing a curing agent, comprising the following steps:
a. Providing citric acid and an alkyl or alkenyl alcohol in a suspension;
b. Mixing and partially esterifying the suspension under reflux conditions to form a monoalkyl or monoalkenyl citrate curing agent substantially free of trialkyl or trialkenyl citrate;
wherein the alkyl alcohol is ethanol, n-propanol or n-butanol; and
wherein the alkenyl alcohol is allyl alcohol.

In another embodiment, this reaction product having free carboxylic acids present in the curing agent is made to react with epoxy monomers to produce thermoset polymers. These polymers may be used to produce various composite materials, like reinforced natural fibers.

In yet other embodiments, biobased curing agents and/or bioresins are also provided.

### BRIEF DESCRIPTION OF THE DRAWINGS

FIG. 1: Calibration curves for citric acid, di- and tri-butyl citrates, and residual butanol

FIG. 2: Calibration curves for citric acid, diethyl, dipropyl and dihexyl citrates

FIG. 3: A butyl citrate mixture separation

FIG. 4: Gradient profile of mobile phase for separation of citric acid, mono-, di- and trialkyl citrates mixtures: ethyl, propyl, butyl and hexyl citrates

FIG. 5: Citric acid conversion into their propyl esters at various mole ratios of n-propanol

FIG. 6: Citric acid conversion into butyl esters at various mole ratios of citric acid: n-butanol

FIG. 7: Citric acid and n-butanol reaction: At the beginning (left) & at the end of the reaction (right).

FIG. 8: Miscibility between alkyl citrate and epoxidized plant oil.

FIG. 9: Natural fiber biocomposite from epoxidized hemp oil and CC-butyl-E (upper) using a hot press, and resin cured tack-free at room temperature in an aluminum pan (lower).

FIG. 10: Bioresins prepared from epoxidized linseed oil and propyl citrate at 25° C.

FIG. 11: A representative load (N) vs. Deflection (mm) graph for biocomposite specimen prepared according to ASTM D7264/D7264M-15.

FIG. 12: Dynamic mechanical analysis of bioresins prepared from EHO, ELO and ethyl, propyl and butyl citrates - Storage (G′) , loss (G″) modulus, and Tan δ.

FIG. 13: Dynamic mechanical analysis of biocomposites prepared from EHO, ELO and ethyl, propyl and butyl citrates — Storage (G′), loss (G″) modulus, and Tan δ.

### DETAILED DESCRIPTION

As mentioned above, the invention relates to the surprising finding that many of the citric acid alkyl ester (CC-alkyl-E) reaction products form a single phase that is substantially or fully miscible with plant-oil-epoxides. CC-alkyl-E reaction products can be optimized for their levels of carboxylic acid functionality and properties of the resulting thermoset polymer, after reaction with plant-oil-epoxides.

### Definitions

As used herein, the following terms have the meaning defined below, unless the context indicates otherwise.

Resin as used herein means a viscous liquid or noncrystalline material with the potential to form a polymer. Resins can be either of natural origin like a gummy semisolid material from certain tree secretions or synthetically derived substances.

In one embodiment, petroleum feedstocks may be transformed into resins. In another embodiment, biobased feedstocks may be transformed into precursors of biobased materials. Resins can be divided into two broad categories: thermoplastic or thermoset resins. In one embodiment, the word “resin” refers to monomers and sometimes to cured materials, while the word “plastic” refers to polymers. Resins can be of 14 major types as listed in Table 1.

TABLE 1
Types of Resin Monomers

| Types of | |
|---|---|
| resins | Commercial resin names |
| Polyester | a. Polyethylene Terephthalate |
| | (PET or PETE) |
| | b. Polybutylene Terephthalate |
| | (PBT) |
| Unsaturated | Unsaturated polyester resin |
| Polyester | |
| Vinyl ester | Vinyl ester resin |
| Phenolic | Phenol formaldehyde (PF): |
| | a. Novolacs |
| | b. Resoles |
| Alkyd | Alkyd resins |
| Polycarbonate | a. Lexan |
| | b. Makrolon |
| Polyamide | a. Nylon |
| | b. Kevlar |
| Polyurethane | Polyurethane resins |
| Silicone | Silicone resins |
| Epoxy | Epoxy resins |
| Polyethylene | a. Low density (LDPE) |
| | b. High density (HDPE) |
| | c. Ultra-high molecular weight |
| | (UHMW-PE) |
| Acrylic | Acrylic resins |
| Polystyrene | Polystyrene resins |
| Polypropylene | Polypropylene resins |

Vegetable oil as used herein means plant oil extracted from oil seeds. In one embodiment, vegetable oils are refined triglycerides of fatty acids where the fatty acids may include saturated or unsaturated fatty acids.

In one embodiment, vegetable oil includes unsaturated fatty acids. In another embodiment, vegetable oils are selected from those oils listed in Table 2. Oil unsaturation levels can be measured by the absorption of iodine or iodine value (IV) and various plant oils have different IVs as listed in Table 2.

TABLE 2
Iodine value of Plant Oils

| | Iodine value |
|---|---|
| Name of plant oil | (gI₂/100 g) |
| Olive | 75-94 |
| Peanut | 80-106 |
| Karanja (Pongamia) | 89 |
| Rapeseed | 94-120 |
| Cottonseed | 90-140 |
| Corn | 103-140 |
| Sunflower | 110-143 |
| Soybean | 117-143 |
| Hemp seed | 155-167 |
| Linseed | 168-204 |
| Camelina | 140-152 |

In one embodiment, the IV of an oil is defined as the number of grams of iodine absorbed by 100 g of oil.

In another embodiment, the unsaturation is used to functionalize or to make more reactive monomers via many chemical transformation routes. In yet another embodiment, functionalization can occur via an epoxidation reaction.

In one embodiment, the greater the IV value, the higher is the content of epoxidation of that oil.

In another embodiment, for instance, hemp oil, flax oil or linseed oil offers potentially better feedstocks for resin production.

In one embodiment, for instance, hemp oil offers potentially better feedstocks for resin production,

In one embodiment, bioresins are potential replacements for synthetic resins.

In another embodiment, vegetable oils are biobased epoxy monomers.

Curing agent as used herein refer to short chain multifunctional compounds categorized by their functional groups: carboxylic acid, amine, amide, anhydride, sulfide, and the like that are able to crosslink with resins. Curing agents include, for instance, crosslinking agents, anionic or cationic initiators or hardeners, and the like.

In one embodiment, these multifunctional compounds may be refered to as hardeners.

In one embodiment, these multifunctional compounds are amenable to crosslinking with resin monomers.

In one embodiment, the resin monomer is an epoxy monomer.

In another embodiment, the epoxy monomer is plant based.

In yet another embodiment, the epoxy monomer is derived from hemp seed oil or hemp oil.

In one embodiment, these multifunctional compounds can be derived from nonrenewable or renewable sources.

In one embodiment, these multifunctional compounds are derived from renewable sources.

In another embodiment, curing agents are selected from those listed in Table 3.

TABLE 3
Classification of curing agents

| Type of | | Functional |
|---|---|---|
| curing agents | Name of curing agents | group |
| Active | Polybasic acids | —COOH |
| hydrogen | Acid anhydrides | (CH₃CO)₂O— |
| containing | Polythiols | —SH |
| compounds | Polyamines | —NH₂—NH—NH₂— |
| | Polyols | —OH |
| Anionic and | Anionic: | 1. Li, Na, K, Cs |
| Cationic | 1. Alkali metal | 2. Pyridine, |
| initiators | derivatives | Amines etc. |
| | 2. Organic compounds | NH3—BF3 |
| | (bases) | |
| | Cationic: | |
| | 1. Complexes of BF₃ | |
| Reactive | Phenol | C₆H₅—OH |
| crosslinkers | Urea (carbamide) | CO(NH₂)₂ |
| | Formaldehyde | —CHO |
| | Melamine | —C₃H₆N₆ |

Aromatic amines are widely used curing agents which are derived from petroleum resources but are also potential human carcinogens and are not useful in the context of the present invention.

In one embodiment, multifunctional and biobased curing agents are compatible with the intended plant oil epoxide for the manufacture of a thermoset material with specified properties.

In one embodiment, compatibility with intended plant oil epoxide refers to the ability of curing agents to be miscible with the plant oil epoxides without requiring added solvent to enhance its miscibility.

In another embodiment, compatibility refers to being biobased in nature, non-toxic, economical and derived from sustainable sources.

In yet another embodiment, non-toxic and biobased food grade carboxylic acids may be used as curing agents as listed in Table 3.

In one embodiment, the listed (Table 3) curing agents do not meet all the requirements of compatibility but their derivates may meet all requirements.

In one embodiment, the present invention is directed to the preparation of curing agent derivatives that meet all the requirements of compatibility for reaction with intended plant oil epoxides.

In another embodiment, a food grade carboxylic acid is citric acid.

In one embodiment, there are many sources to obtain biobased curing agents. For instance, plant oils, lignins, rosin derivatives, biobased phenols, acids, and anhydrides.

In one embodiment, the biobased curing agent is citric acid. Citric acid (2-hydroxy-propane-1,2,3-tricarboxylic acid) is synthesized from various simple sugars by fermentation with Aspergillus niger mycelial fungi.

In one embodiment, citric acid is hygroscopic and highly water soluble (160.8 g of citric acid/100g H₂O at 25° C. for a saturated solution of citric acid in water).

In another embodiment, citric acid is incompatible for use with hydrophobic plant oils, however, in yet another embodiment, citric acid is a candidate for derivatization before use with hydrophobic plant oils.

In one embodiment, the derivatization reaction is a partial esterification leading to the formation of monoalkyl citrates so as to preserve two carboxylic acids for curing epoxide vegetable or plant oils.

As used herein the term epoxides refers to uncured epoxide monomers while a cured resin refers to epoxides crosslinked into a rigid thermoset material. In one embodiment, a multifunctional compound crosslinks the epoxidised triacylglycerols into a three-dimensional polymer network via oxirane ring opening reactions. The process of crosslinking is referred to as curing of the epoxides, to make rigid bioresins or thermoset materials.

In one embodiment, plant oils are the most attractive resource having functionality for the synthesis of bioresins. However, sustainable bioresin production without the use of toxic chemicals and solvents makes finding alternate ways challenging.

As used herein the term biocomposite refers to a material prepared by combining a biobased or a non-biobased polymer matrix with natural fibers as a reinforcement component. Since two completely different components are involved in forming a biocomposite, its properties are influenced by interfacial adhesion, structure, and their bonding mechanisms.

As used herein the term natural fibers refers to fibers from agriculture waste or are byproducts of certain crops at low cost. Natural fibers may also be sustainable materials which could be used to manufacture various products. Certain natural fibers like flax straw, hemp, coir, and bamboo have the necessary high modulus-to-density and strength-to density ratios as well as high availabilities, for them to be considered as potential feedstocks to manufacture biobased products, including biocomposites. Other short natural fibres are often waste products but can still be used in biocomposites as fillers.

In one embodiment, natural fibers have the potential to replace synthetic fibers that are currently used in many composite materials.

In another embodiment, the surface chemistry of natural fibers can be derivatized to overcome their typical hydrophilic nature, which can be a challenge for their utilization in biocomposites containing less polar or hydrophobic polymers.

In one embodiment, the properties of natural fibers vary based on their chemical composition and structure. The chemical composition of natural fibers is shown in Table 4.

TABLE 4
Natural Fiber - Type and Composition

| Natural | Type/ | Chemical composition | | |
|---|---|---|---|---|
| fiber | Origin | Cellulose (%) | Hemicellulose(%) | Lignin (%) |
| Hemp | bast | 68 | 15 | 10 |
| Flax | bast | 71 | 18.6-20.6 | 2.2 |
| Sisal | leaf | 65 | 12 | 9.9 |
| Coir | fruit/seed | 32-43 | 0.15-0.25 | 40-45 |
| Ramie | bast | 68.6-76.2 | 13-16 | 0.6-0.7 |
| Bamboo | grass | 26-43 | 30 | 21-31 |
| Sugarcane | stalk | 55.2 | 16.8 | 25.3 |
| bagasse | residue | | | |
| Jute | bast | 61-71 | 14-20 | 12-13 |
| Abaca | leaf | 56-63 | 20-25 | 7-9 |
| Kenaf | bast | 72 | 20.3 | 9 |

As used herein, “substantially miscible” means that the substance is at least 50%, preferably at least 60%, and more preferably at least 80% soluble in the plant-oil-epoxide.

“Alkyl” means a saturated or unsaturated straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of saturated straight chain alkyl groups include, but are not limited to, methyl, ethyl, n-propyl, n-butyl, n-pentyl-, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups.

In some embodiments, alkyl is a C₁₋₆ alkyl optionally substituted with one or more substituents.

“Isomer” is used herein to encompass all chiral, diastereomeric or racemic forms of a structure (also referred to as a stereoisomer, as opposed to a structural or positional isomer), unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

The singular forms “a,” “an,” and “the” include the plural reference unless the contextclearly dictates otherwise. The term “and/or” means any one of the items, any combination ofthe items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variationfrom 45 to 55 percent. For integer ranges, the term “about” can include one or two integers greater than and/or less than a recited integer at each end of the range. Unless indicated otherwise herein, the term “about” is intended to include values and ranges proximate to the recited value or range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “between”, “up to”, “at least”, “greater than”, “less than”, “more than”, “or more”, and the like, include the number(s) recited and such terms refer to ranges that can be subsequently broken down into sub-ranges as discussed above. In the same manner, all ratios recited hereinalso include all sub-ratios falling within the broader ratio.

### Curing Agents

As detailed above, the present disclosure provides methods for preparing curing agents with significant improvement in compatibility with epoxy plant oils for large scale manufacture of bio-based thermoset resins. Accordingly, the present disclosure provides in one embodiment a method of preparing a curing agent, comprising the following steps:
a. providing citric acid and an alkyl or alkenyl alcohol in a suspension;
b. mixing and partially esterifying the suspension under reflux conditions to form a monoalkyl or monoalkenyl citrate curing agent substantially free of trialkyl or trialkenyl citrate;
wherein the alkyl alcohol is ethanol, n-propanol or n-butanol; and
wherein the alkenyl alcohol is allyl alcohol.

In one embodiment, the suspension upon mixing becomes a single phase in the absence of any added solvent.

In another embodiment, the curing agent is substantially free of dialkyl or dialkenyl citrate.

In one embodiment, the alkyl alcohol is ethanol.

In another embodiment, the alkyl alcohol is n-propanol.

In yet another embodiment, the alkyl alcohol is n-butanol.

In one embodiment, esterifying the citric acid occurs above the boiling point of the alcohol and below about 130° C.

In one embodiment, the ratio of citric acid to alcohol is between about 1:0.5 (mole/mole) to about 1:4 (mole/mole).

In another embodiment, the ratio of citric acid to alcohol is between about 1:0.5 (mole/mole) to about 1:2 (mole/mole).

In one embodiment, the monoalkyl citrate is present in about 60 wt % to about 85 wt % in presence with unreacted citric acid.

In another embodiment, the monoalkyl citrate is present in about 75 wt % to about 85 wt % in presence with unreacted citric acid.

In one embodiment, the present disclosure provides for a curing agent where the curing agent is prepared by the methods described above.

In one embodiment, is provided a method of making a thermoset polymer comprising reacting free carboxylic acids present in a monoalkyl citrate curing agent with epoxy monomers.

In another embodiment, the monoalkyl citrate is miscible with epoxy monomers at room temperature.

In one embodiment, the monoalkyl citrate and epoxy monomers remain a single phase mixture at room temperature.

In another embodiment, the epoxy monomers are derived from unsaturated triglycerides in plant oil.

In one embodiment the plant oil is hemp oil.

In another embodiment, the present disclosure provides for a thermoset polymer prepared according to the methods described above.

In one embodiment is provided a composite material comprising the thermoset polymer of the present invention.

In another embodiment, the present disclosure provides a biocomposite material comprising a natural fibre and a biobased resin, wherein the biobased resin is prepared from a biobased curing agent and a plant oil epoxy; wherein the biobased curing agent is prepared from bioethanol; and wherein the biocomposite material is substantially made from renewable sources.

In other embodiment, the alcohol of the present disclosure may comprise an alkyl alcohol, having twenty carbon atoms or less, preferably six carbon atoms or less, and more preferably 4 carbon atoms or less. The alcohol may be a primary, secondary or tertiary alcohol.

In another embodiment, the alcohol of the present disclosure may be biobased, like ethanol.

In some embodiments, the selection of the citric acid-to-alcohol mole ratio used in the reaction is at least partly dependent on the alcohol selected. The citric acid-to-alcohol mole ratio used in the reaction should be greater than about 1:0.1.

In other embodiments, the citric acid-to-alcohol mole ratio used in the reaction may vary from between about 1:0.3 to about 1:20.

In other embodiments, the citric acid-to-alcohol mole ratio usedin the reaction may vary from between about 1:1 to about 1:10. The actual citric acid to alcohol ratio in the reaction product is lower than the input ratios—for example, the final product ratio would be 1:0.33 mol/mol citric acid/alcohol if allof the reaction product is a mono-ester.

In one embodiment, CC-alkyl-E reaction products contain significant levels of unreacted citric acid, yet form a single phase that is substantially or fully miscible with plant-oil-epoxides. CC-alkyl-E reaction products can be optimized for their levels of carboxylic acid functionality and properties of the resulting thermoset polymer, after reaction with plant-oil-epoxides.

In one embodiment, the reaction temperature should be below the decomposition temperature of citric acid, which is above 130° C., and above the boiling point of the alcohol so that the reaction is performed under reflux conditions. Therefore, in some embodiments, the reaction temperature may be between about 90° C. and 130° C. The esterification reaction generates water as a byproduct and reflux conditions discourages the formation of diester andtriester citrate species. Therefore, in some embodimens reflux conditions are utilized to minimize the production of these undesirable citrates.

The reaction product will consist essentially of monoalkyl citrate, dialkyl citrate, and possibly trialkyl citrate and some unreacted citric acid and alcohol. In some cases, unreacted alcohol may be removed, such as by distillation. The dialkyl and trialkyl citrates are not desirable, and reaction conditions should be varied to minimize their yield. In some embodiments, the reaction product will comprise at least about 40% monoalkyl citrate.

In some embodiments, the monoalkyl citrate is present in about 50%, and in some other embodiments, the monoalkyl citrate is present in greater than about 75%.

In other embodiments, the reaction product is substantially free of trialkyl citrate (less than about 5%) and has less than 30% dialkyl citrate.

In some embodiments, the reaction product has less than 20% dialkyl citrate.

In some embodiments, using n-butanol as the alcohol, the reaction may be optimized to obtain the maximum yield of monobutyl citrate, the lowest possible yield of dibutyl citrate and no formation of tributyl citrate. N-butanol has a boiling point of 117.7° C., therefore, the reaction temperature may be in the range of about 118° to 130° C.

The properties of a cured bioresin material depend on the oil epoxide structure, the curing agent and the curing parameters employed in its preparation. Thus, manufacturing of bioresin based materials with targeted properties can be achieved by selecting appropriate epoxide monomers, curing agents, and curing condition or processes. For instance, material prepared by using the shorter carbon chain length succinic acid (C4) as a curing agent is rigid compared to material prepared by using longer carbon chain length sebacic acid (C10).

All reagents, for which the synthesis is not described in the experimental part, are either commercially available, or are known compounds or may be formed from known compounds by known methods by a person skilled in the art. The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC using methods as described.

Chemical names were generated using the ChemDraw naming software (Version 17.0.0.206) by PerkinElmer Informatics, Inc. In some cases, generally accepted names of commercially available reagents were used in place of names generated by the naming software.

### EXAMPLES

### General Methods

Citric acid (2-hydroxypropane-1,2,3-tricarboxylic acid, MW=192 g/mol) was purchased from Jungbunzlauer Canada Inc. and ground it into a fine powder by Fitz Das06 hammer mill and passed through 0.25 mm sieve. Synthesis grades of ethanol, n-propanol, n-butanol, and n-hexanol were purchased from Sigma. Analytical ACS grade deionized water, acetonitrile, formic acid, and methanol were purchased and used in HPLC/ELSD.

### Example 1

### Synthesis of Alkyl Citrates

All of the following alkyl citrates were synthesized via a solvent- and catalyst-free esterification of citric acid with four different alcohols: ethanol, n-propanol, n-butanol and n-hexanol. The esterification reaction was performed under reflux conditions which kept the reactant alcohol and byproduct water in the reaction mixture. Removal of byproduct, water, from the esterification reaction enhanced the reaction rate towards formation of the thermodynamically possible products of di- and trialkyl citrates. However, with the water byproduct staying in the reaction mixture, the reaction between the remaining carboxylic acids and alcohols to form an ester bonds was contained to forming the monoalkylcitrate.

For alkyl citrates to be curing agent in the present invention, the product must have to have at least two functional groups which would be available to react with an epoxy group to form a crosslinked network. A trialkyl citrate has no available carboxylic acids so cannot participate in the crosslinking or curing reaction. Hence, intermediate samples were analyzed to determine the citric acid conversion and the extent of mono, di and trialkyl esters formation.

General scheme 1 illustrates the goal of synthesizing alkyl citrates as curing agents so they remain multifunctional and miscible with the plant oil epoxides.

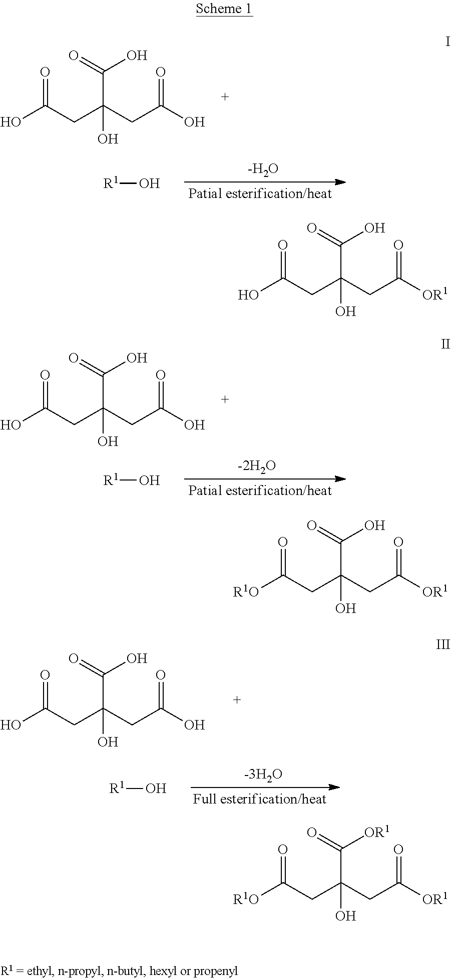

### Optimization of Synthetic Parameters

The reaction progress at various citric acid to alcohol mole ratios was conducted, 200 grams of citric acid was used, and intermediate samples were collected and analyzed. The citric acid consumption and the monoalkyl citrate formation continued up to certain point then their consumption/synthesis progress become constant. It was surprisingly found that the highest functionality was achieved at the minimum reaction time at which the reaction mixture become transparent.

Noting this transparency of reaction mixture, the remaining two alkyl citrates, ethyl and hexyl, were synthesized by taking 20 grams of citric acid with various amounts of alcohols. Citric acid esterification was performed under reflux condition at 90° C. with ethanol which has a boiling point of 78° C. The reaction temperature was determined by considering two factors: the boiling point of the alcohol and the decomposition temperature of citric acid, which occured above 130° C. Therefore, the reaction temperatures for all four alcohols used in this study were less than 130° C. Lower reaction temperatures may be possible, but they are avoided due to the limited solubility of citric acid in alcohols at lower temperatures, which also led to higher reaction times. Simultaneously, the reaction time was minimized because with longer reaction times led to formation of di- and trialkyl citrate compounds formation as described in the individual examples.

Citric acid esterification with propanol was performed at 110° C. with n-propanol having a boiling point of 97° C. under reflux condition. For the reaction with n-butanol, the boiling point of n-butanol is 117° C., so a 130° C. reaction temperature was selected.

For the citric acid esterification with hexanol, despite the boiling point of hexanol being 157° C., reaction temperatures of 120° C. and 130° C. were selected to avoid citric acid decomposition. All the intermediate and final alkyl citrate samples were evaporated at 100° C. under vacuum pressure in a rotary evaporator to remove byproduct water and unreacted alcohols. The residual alcohol content in these samples were below 0.3%.

Quantification of Alkyl Citrates with HPLC-ELSD

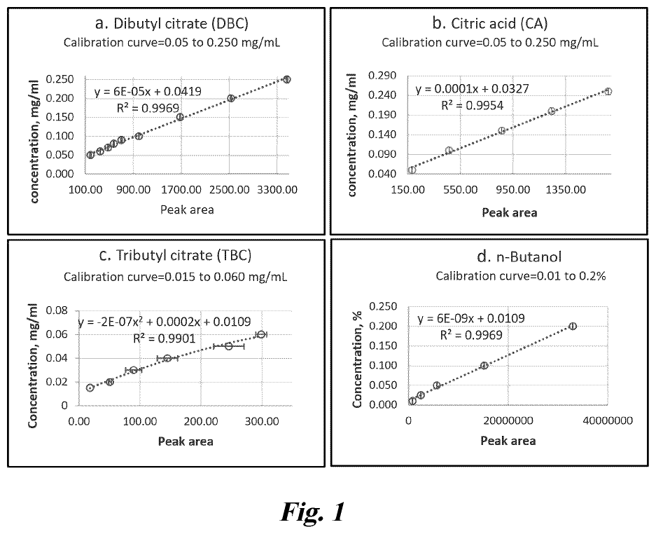

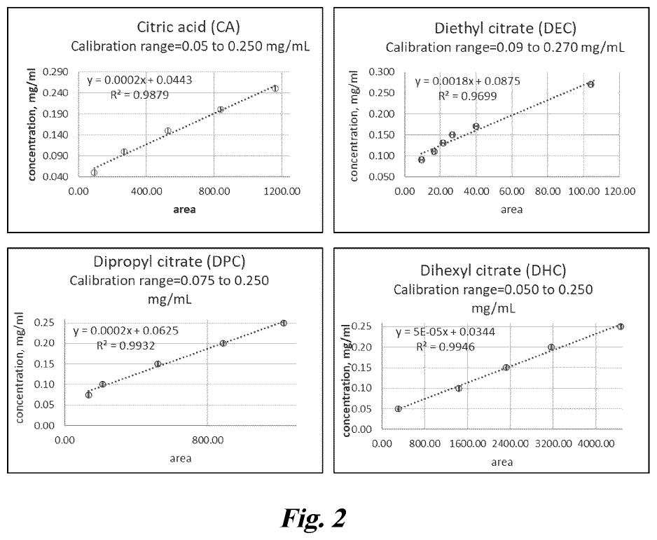

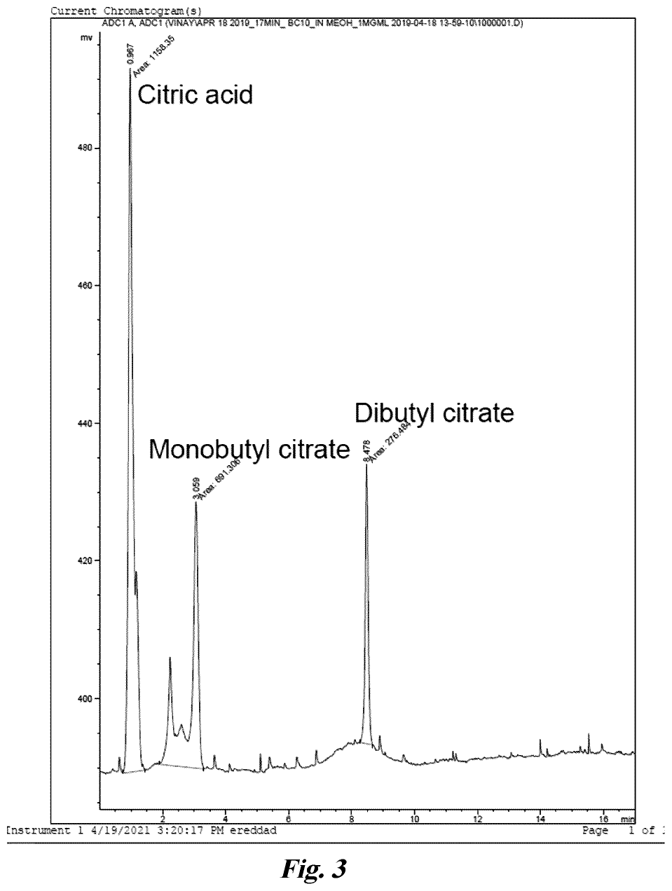

In all the following procedures, the Evaporative Light Scattering Detector (ELSD) was used to quantify, measure and establish the analytical method to calibrate the alkyl citrates. First, the ELSD detector parameters, tube temperature and nitrogen gas flow rate, were optimized. It was found that the tube temperature of 30° C. and nitrogen gas flow rate of 1.5 L/min were optimum to obtain the highest peak area of citric acid, di- and trialkyl citrates. The detector gain factor of 16 used and impactor set at OFF position. Second, the calibration range was determined by considering the estimated composition of the citrate mixtures fall within these concentrations as long as the data are linear. All these calibration data of citric acid and various alkyl citrates are presented in the FIGS. 1 and 2. A butyl citrate mixture separation is depicted in FIG. 3.

In order to prepare a dibutyl citrate standard for quantification purposes, butyl citrate was separated from the citric acid, mono-, and tributyl citrates by liquid-liquid extraction procedure by using diethyl ether as solvent at various pH levels. First, the mixture was dissolved in diethyl ether and the solution pH increased to 8.0. Then the aqueous phase separated from the organic phase which contains tributyl citrate. Finally, the solution pH was further decreased to 2.0 and the aqueous phase removed containing citric acid and monobutyl citrate. Tributyl citrate was commercially available with greater than 99% purity and used as is to obtain a calibration curve. Calibration curves were obtained as depicted in FIG. 1 for citric acid, di-, and tributyl citrates. The monobutyl citrate amount was quantified by subtracting the sum of the quantified amounts of citric acid, di- and tributyl citrates.

Diethyl citrate was purified by liquid-liquid extraction and used to obtain the calibration curve as depicted in FIG. 2. Under optimized reaction conditions, the formation of trialkyl citrate did not significantly occur, so it was not necessary to quantify triethyl citrate in such ethyl citrate mixtures.

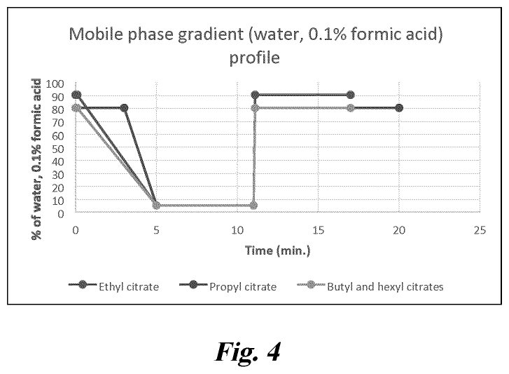

Since the various chain length alcohols were used to synthesized alkyl citrates, each alkyl citrate was separated in a C18 column by slight modification of the mobile phase gradient, as illustrated in FIG. 4. Water and acetonitrile buffers were used as mobile phase solvents.

### Example 2

### Synthesis of Ethyl Citrate

The selection of the citric acid-to-alcohol mole ratios used in the preparation are at least partly dependent on the alcohol selected. First, the realistic ratio of citric acid to ethanol ratio was identified by considering the reaction time not exceeding 5 hours because at a greater reaction time, di- and trialkyl citrates started to form. To keep the reaction time at a minimum, a higher amount of alcohol can be used but too much excess alcohol contributed to formation of di- and trialkyl citrates. A citric acid to ethanol equimolar ratio, 1:1, was not suitable because the citric acid was not fully dissolved at 90° C., and the mixture did not become one phase after 6 hours of reaction.

TABLE 5
Ethyl citrate formation under various ethanol mole ratios

| Citric | | | Free | | | | Average |
|---|---|---|---|---|---|---|---|
| acid to | Reaction | Reaction | citric | Monoethyl | Diethyl | Triethyl | carboxylic |
| ethanol | time | temperature | acid | citrate | citrate | citrate | acid |
| (mol:mol) | (min.) | (° C.) | (%) | (%) | (%) | (%) | functionality |
| 1:1 | 360 | 90 | Reactants did not become | | | | n/a |
| | | | homogeneous | | | | |
| 1:2 | 77* | | 34.3 | 65.7 | 0.0 | 0.0 | 2.3 |
| 1:2 | 120 | | 17.2 | 82.8 | 0.0 | 0.0 | 2.2 |
| 1:2 | 180 | | 8.9 | 73.6 | 17.5 | 0.0 | 1.9 |

### Example 3

### Synthesis of Propyl Citrate

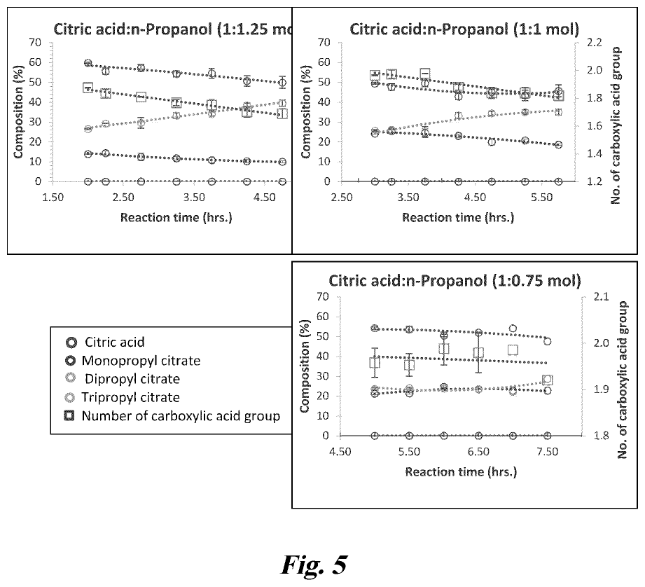

FIG. 5 depicts the formation of propyl citrates at three different mole ratios of citric acid to n-propanal. In each case, the first point on the graph is at the time when the reaction mixture becomes a single phase. In each case, it can be seen in FIG. 5 that dipropyl citrate is already formed at these minimum reaction times. For crosslinking applications, the presence of di- and trialkyl citrate is not suitable because they inhibit the polymer chain growth. Therefore, additional propyl citrate formulations were prepared to avoid formation of di- and tripropyl citrates as depicted in Table 6.

Dipropyl citrate was not formed when citric acid to n-propanol ratio of 1:2 used and an average carboxylic acid functionality of 2.3 was achieved as indicated in Table 6. The dipropyl citrate amount increased as the alcohol amount increased, and it surprisingly became 0 at the citric acid to propanol ratio of 1:2. Then, at the 1:2.5 mol ratio, the amount of dipropyl citrate increased again to 17.5% as this is not only dependent on the alcohol amount but was found to also depend on reaction time, rate and solubility of the reactants and products.

TABLE 6
n-Propyl citrate formation under various n-propanol mole ratios

| Citric | | | Free | Mono- | | | Average |
|---|---|---|---|---|---|---|---|
| acid to | Reaction | Reaction | citric | propyl | Dipropyl | Tripropyl | carboxylic |
| npropanol | time* | temperature | acid | citrate | citrate | citrate | acid |
| (mol:mol) | (min.) | (° C.) | (%) | (%) | (%) | (%) | functionality |
| 1:1.50 | 77 | 110 | 24.7 | 57.6 | 16.7 | 0.0 | 2.1 |
| 1:1.75 | 50 | | 21.5 | 60.3 | 17.2 | 0.0 | 2.0 |
| 1:2.00 | 50 | | 29.1 | 69.9 | 0.0 | 0.0 | 2.3 |
| 1:2.50 | 35 | | 20.2 | 61.3 | 17.5 | 0.0 | 2.0 |

### Example 4

### Synthesis of Butyl Citrate

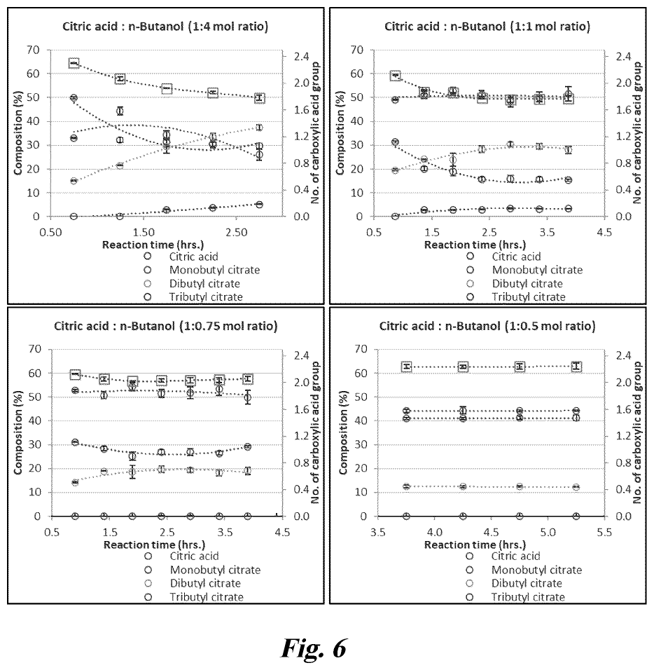

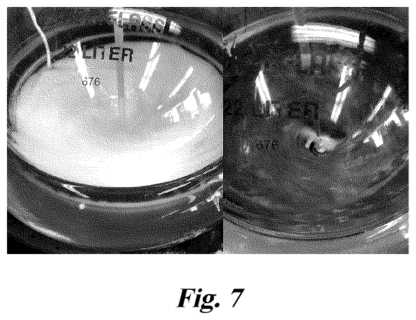

Four different citric acid to n-butanol mol ratios were used and the reaction progress monitored as depicted in FIG. 6. Tributyl citrate was not formed when limiting amounts of n-butanol were used for the reaction, as seen with 0.50 and 0.75 moles of butanol per mole of citric acid. At these citric acid-to-n-butanol mole ratios, dibutyl citrate formation of between 10 and 15 occurred %. For all four different mole ratios, the monobutyl citrate content is comparatively similar (between 50 to 55%) and as the reaction progresses (FIG. 7) monobutyl citrate amounts stabilized which indicated that the reaction establishes to its equilibrium. The citric acid conversion also reaches an equilibrium state for all mole ratios except for the case of mole ratio of 1:4, where excess n-butanol is available to be used in the reaction, and free citric acid continues to be consumed.

It can be concluded that the n-butanol amount and the reaction time affects the carboxylic acid functionality of the citrate mixture which can be tuned to achieve desired functionality. After determining the effects of esterification reaction parameters onto the citric acid conversion and mono, di-, and tributyl citrates formation, five additional butyl citrate formulations were prepared to determine their viscosities and how they are corelated with the composition of the mixture as depicted in the Table 7. It can be seen that formulation 3, which has the highest monobutyl citrate concentration, has the lowest viscosity. However, the overall viscosity is affected by all components of the butyl citrate mixture. The viscosities are found to be drastically reduced at 50° C., specifically for formulation no. 3. In contrast, formulation no. 4 has the lowest amount of monobutyl citrate and highest amount of free citric acid, also exhibits the highest viscosity.

TABLE 7
Viscosities of butyl citrate formulations

| | CA | MBC | DBC | TBC | Viscosity (Pa · S) | | | | | |
|---|---|---|---|---|---|---|---|---|---|---|
| | (%) | | | | 25° C. | 30° C. | 35° C. | 40° C. | 45° C. | 50° C. |
| BC-1 | 17.3 | 49.4 | 29.0 | 3.4 | 3264 | 1887 | 1463 | 1403 | 1391 | 1402 |
| BC-2 | 28.7 | 51.0 | 19.3 | 0.0 | 10825 | 6956 | 4439 | 2073 | 1562 | 1481 |
| BC-3 | 22.0 | 63.1 | 13.8 | 0.0 | 3224 | 1207 | 488 | 209 | 97 | 48 |
| BC-4 | 42.0 | 44.4 | 12.4 | 0.0 | 23929 | 18336 | 13970 | 11523 | 9266 | 5017 |
| BC-5 | 37.9 | 47.3 | 13.6 | 0.0 | 17159 | 5665 | 2069 | 820 | 349 | 157 |

### Example 5

### Synthesis of Hexyl Citrate

Two reaction temperatures were used and various citric acid to n-hexanol mol ratios were used to synthesize hexyl citrate mixtures as depicted in Table 8. It was found that dihexyl citrate was formed at both temperatures and the average carboxylic acid group functionality is less than 2.0 for all formulations. Since the curing agent must have at least two functional groups in order to form a network of crosslinks, these formulations with less than 2 functionalities are not suitable.

TABLE 8
n-Hexyl citrate formation under various n-hexanol and mol ratios

| Citric | | | Free | | | | Average |
|---|---|---|---|---|---|---|---|
| acid to | Reaction | Reaction | citric | Monohexyl | Dihexyl | Trihexyl | carboxylic |
| nhexanol | time | temperature | acid | citrate | citrate | citrate | acid |
| (mol:mol) | (min.) | (° C.) | (%) | (%) | (%) | (%) | functionality |
| 1:1 | 282 | 120 | 14.7 | 52.8 | 27.2 | 5.4 | 1.8 |
| 1:1.25 | 210 | | 10.3 | 47.5 | 36.1 | 6.0 | 1.6 |
| 1:0.75 | 96 | 130 | 20.7 | 56.1 | 20.1 | 3.0 | 1.9 |
| 1:1 | 120 | | 17.2 | 54.2 | 24.9 | 3.7 | 1.9 |
| 1:1.25 | 60 | | 16.1 | 58.9 | 22.2 | 2.8 | 1.9 |

### Example 6

### Synthesis Of Bioresins

The objective of synthesizing these partial esters of citric acid was to prepare a plant oil epoxide miscible curing agent. This miscible curing agent made possible the synthesis of fully biobased epoxy resins without the use of solvent. These citrates were miscible with the plant oil epoxides as depicted in Table 9.

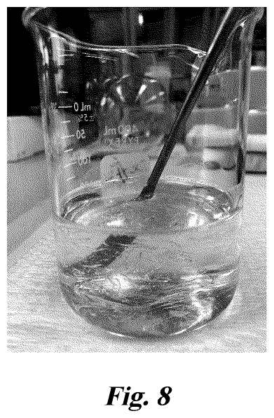

A stable single phase mixture is seen in FIG. 8 when at room temperature the epoxidized plant oil is mixed with CC-butyl-E, for instance.

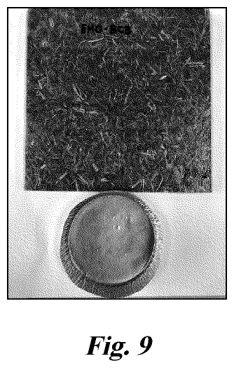

The viscosity of the resin mixture containing oil epoxide and curing agent CC-butyl-E at room temperature is about 5 Pa. S, it has sufficiently low viscosity to adequately penetrate natural fibers in making biocomposite materials. The pot life of such an epoxy resin is about 1.5 h. Later, the mixture was placed in a aluminum pan at room temperature and cured until it reached a tack-free state, which is seen as a clear resin in the aluminum tray shown in FIG. 9.

TABLE 9
Miscibility of biobased curing agents with plant oil epoxides

| | | | | | Miscible |
|---|---|---|---|---|---|
| | Composition of the curing agent | | | | with the |
| Name of | Free | Monoalkyl | Dialkyl | Trialkyl | plant oil |
| the curing | citric acid | citrate | citrate | citrate | epoxide* |
| agent | (%) | (%) | (%) | (%) | at 50° C. |
| Ethyl | 36.8 | 63.2 | 0.0 | 0.0 | No |
| citrate-1 | | | | | |
| Ethyl | 17.2 | 82.8 | 0.0 | 0.0 | Yes |
| citrate-2 | | | | | |
| Propyl | 22.0 | 60.0 | 18.0 | 0.0 | Yes |
| citrate-1 | | | | | |
| Propyl | 29.1 | 69.9 | 0.0 | 0.0 | Yes |
| citrate-2 | | | | | |
| Butyl | 22.0 | 63.1 | 13.8 | 0.0 | Yes |
| citrate-1 | | | | | |
| Butyl | 41.3 | 44.7 | 12.9 | 0.0 | No |
| citrate-2 | | | | | |
| Hexyl | 20.7 | 56.1 | 20.1 | 3.0 | Yes |
| citrate-1 | | | | | |
| Hexyl | 10.3 | 47.5 | 36.1 | 6.0 | Yes |
| citrate-2 | | | | | |

### Example 7

Optimisation of the epoxy to curing agent ratios was achieved through the measurement of the glass transition temperature through differential scanning calorimetry (DSC) measurements on epoxide-curing agent mixtures and cured resins. Then, utilizing these optimum ratios, bioresins and natural fiber biocomposites were prepared, and their mechanical and thermomechanical properties measured using flexure tests and dynamic mechanical analysis (DMA). One of the goals was to prepare a miscible plant-oil-epoxide-curing agent mixture that can impregnate the natural fiber without the use of solvent. Ultimately, the purpose of the testing was to determine the comparative mechanical and thermomechanical performances of the newly synthesized bioresins for biocomposite applications.

### Materials and Methods

The nonwoven natural fibermat with 60% wood, 40% hemp fiber content and density of 2.5 kg/m³ was generously provided by the Biocomposites Group, Drayton Valley, Alberta. The epoxidized hemp and linseed oils were prepared in the lab using hydrogen peroxide and formic acid as catalyst. The double bonds present in the oils were transformed into oxiranes via peroxyacids (R—COO—O—H) formation from formic acid and hydrogen peroxide. The compositions of the citric acid-alkyl esters used in this study are depicted in Table 10. These curing agent formulations were chosen because they are miscible with the plant oil epoxides at 50° C. and have a pot life of about 1 hour.

TABLE 10
Compositions of citric acid-alkyl citrates

| | | | | | Average |
|---|---|---|---|---|---|
| | | | | | carboxylic acid |
| | Composition of CA-alkyl citrates | | | | functionality/mole |
| Propyl | Citric | Monopropyl | Dipropyl | Tripropyl | |
| citrate (PC) | acid (%) | citrate (%) | citrate (%) | citrate (%) | |
| | 29.1 | 69.9 | 0 | 0 | 2.3 |
| Ethyl citrate | Citric | Monoethyl | Diethyl | Triethyl | |
| (EC) | acid (%) | citrate (%) | citrate (%) | citrate (%) | |
| | 17.2 | 82.8 | 0 | 0 | 2.2 |
| Butyl citrate | Citric | Monobutyl | Dibutyl | Tributyl | |
| (BC) | acid (%) | citrate (%) | citrate (%) | citrate (%) | |
| | 22.0 | 63.1 | 13.8 | 0 | 2.1 |

### Determination of Optimum Ratio's

Pure resin without any reinforcement material was used to determine the optimum epoxy to carboxylic acid groups ratios for all epoxide-curing agents formulations. Then, biocomposites were prepared from this resin by utilizing natural fiber as the reinforcement material. The mechanical and thermomechanical properties were compared to determine the effect of each curing agent on the biocomposites' properties. These properties are correlated with characteristics including the % OOC of the epoxides, the curing agent's functionality, and the amount of plasticizing compounds (di-alkyl citrates) present in the curing agent.

### Bioresin Preparation

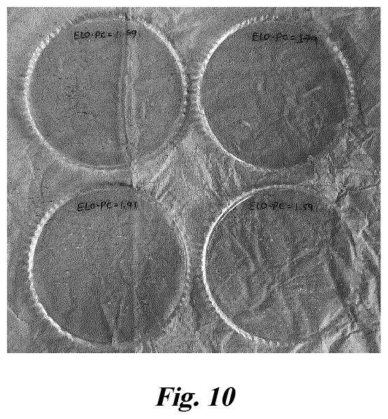

The curing behavior was determined through a DSC method. The epoxide and curing agent were separately heated to 50° C. and then mixed for about 10 minutes to achieve a homogeneous mixture. Then, the mixture was placed in vacuum (25 mmHg) to remove any trapped air bubbles from the bulk liquid at room temperature for about 10 minutes. It was then transferred to an aluminum pan and kept at 25° C. over the next 22 days. Previously, it was observed that such epoxide-curing agent mixtures were cured to become tack-free rigid material withing 48 hours (at 25° C.) so every 10 days the curing progress was measured. FIG. 10 illustrates bioresin samples prepared from epoxidized linseed oil and propyl citrate.

Additionally, bioresin samples were cured at elevated temperature. The epoxide and curing agent mixture was prepared as described above until after degassing under vacuum. Then, the liquid mass was transferred into an aluminum pan, and it was placed in an oven which was set at 25° C. Then the oven temperature increased to 140° C. at a rate of about 2° C/min. The oven temperature was kept isothermal at 140° C. for 1 hour then cooled down to 25° C.

### Butyl Citrate Mixtures

Differential scanning calorimetry is one of the tools used to determine the optimum ratio of epoxide and curing agents by measuring their glass transition temperatures. At the optimum epoxy to carboxylic acid groups ratio, the crosslinking density would be highest and there would remain the lowest number of unreacted components, the epoxy monomers and curing agent. As a result of these two effects, at the optimum epoxy to carboxylic acid groups ratio, where there would be highest crosslinking density, the highest glass transition temperature amongst samples prepared by using different epoxy to carboxylic acid groups ratios would be observed.

Since the curing agents used were synthesized for the first time, the optimum epoxide to curing agent ratio needs to be determined. The epoxide curing agent mixture was prepared as described in the Bioresin Preparation section above and allowed to cure at 25° C. over the next 22 days. The Tg was measured at days 0, 12 and 22. The Tg at day 0 was determined by taking the bulk liquid sample and cured in the DSC as follows. The sample mixture was heated to 160° C. at 2° C/min then held isothermal for 10 min. It was cooled down to −90° C. and the temperature was raised to 160° C. within ±1° C/min. using the modulated mode. At days 12 and 22, samples were taken from the bulk bioresin for measurement of their Tg. The material with the highest crosslinking density would have a higher glass transition temperature (Tg) than the material with a lower crosslinking density. Additionally, the highest Tg can also indicate the lowest amount of unreacted monomer present in the cured sample compared to the material with the lower Tg. Such an unreacted monomers have plasticizing effect which is responsible for the low Tg of the material. By considering these parameters, it can be hypothesized that if the maximum number of epoxy groups are crosslinked via reaction with carboxylic acid groups, then in this case there should be least amount of unreacted monomers/curing agents present in the cured sample.

TABLE 11
Optimum Ep/Ac groups molar ratio for the
epoxidized linseed oil and butyl citrate mixture
Epoxidized linseed oil/Butyl citrate

| | | | | Cured @ |
|---|---|---|---|---|
| Epoxy/Acid | Day 0 | Day 12 | Day 22 | 140° C., |
| (Ep/Ac) groups | Cured at room temperature | | | 1 hrs. |
| molar ratio | Tg0 (° C.) | Tg12 (° C.) | Tg22 (° C.) | Tg (° C.) |
| 1.00 | 18.8 | 23.0 | 21.7 | |
| 1.52 | 26.0 | 33.2 | 34.4 | |
| 1.61 | 29.7 | 32.6 | 36.2 | |
| 1.71 | 31.3 | 37.0 | 43.6 | 37.6 |
| 1.82 | 28.1 | 38.7 | 38.9 | |
| 1.95 | 28.4 | 28.5 | 33.4 | |

This material should have the highest Tg compared to one which has an unbalanced number of epoxy to carboxylic acid groups in the reaction mixture. In the case of the butyl citrate curing agent with epoxidized linseed oil, the epoxide to carboxylic acid group molar ratio of 1.7 is optimum as it has the highest Tg among all other ratios. The highest Tg was also observed at this molar ratio as curing progresses for the day 12 and 22 samples (Table 11).

The same epoxy to carboxylic acid groups molar ratio, 1.7, was used to prepare a bioresin at an elevated curing temperature of 140° C., and this sample has Tg of 37.64° C. which was about 6° C. less than the sample cured at 25° C. for 22 days. This could be due to differences between the curing profiles at room temperature vs 140° C. as the reactive compounds in the bulk mixture have more time to react at room temperature. In contrast, with the higher temperature curing, these reactive components are crosslinked quicker and hence the remaining unreacted monomers do not get enough time to become part of overall crosslinking network.

When the epoxidized hempseed oil was used with the butyl citrate as curing agent (Table 12), the optimum epoxy to carboxylic acid groups molar ratio was determined as 1.5. Thus, the ratio of 1.7 was optimum for the epoxidized linseed oil but 1.5 for epoxidized hempseed oil, which means that the later formulation is less epoxy rich. This could be due to the differences between the curing kinetics of these two epoxidized oils, as it was proven that epoxides with higher % OOC have higher reactivities than those with lower % OOC.

TABLE 12
Optimum Ep/Ac groups molar ratio for the
epoxidized hempseed oil and butyl citrate mixture
Epoxidized hempseed oil/Butyl citrate

| | | | | Cured @ |
|---|---|---|---|---|
| Epoxy/Acid | Day 0 | Day 12 | Day 22 | 140° C., 1 |
| (Ep/Ac) groups | Cured at room temperature | | | hrs. |
| molar ratio | Tg0 (° C.) | Tg12 (° C.) | Tg22 (° C.) | Tg (° C.) |
| 1.00 | 8.9 | 11.8 | 12.5 | |
| 1.40 | 10.0 | 17.5 | 18.3 | |
| 1.49 | 14.3 | 19.8 | 23.0 | 16.6 |
| 1.59 | 10.3 | 16.4 | 21.1 | |
| 1.70 | 11.9 | 20.6 | 21.6 | |
| 1.84 | 9.4 | 12.7 | 14.6 | |

The difference between bioresin's Tg prepared from epoxidized linseed and hempseed oil with the butyl citrate was about 21° C. due to the differences in their % OOC, as 8.3% and 9.8% OOC of EHO and ELO, respectively.

### Example 8

### Propyl Citrate Mixtures

The optimum Ep/Ac ratio of 1.7 was determined for the epoxidized linseed oil and propyl citrate since this ratio results in the maximum Tg for day 0, 12 and 22 (Table 13). The Tg of bioresin prepared by using this ratio of 1.7 and cured at elevated temperature was about 3° C. higher than the sample cured at 25° C. for 22 days.

TABLE 13
Optimum Ep/Ac groups molar ratio for the
epoxidized linseed oil and propyl citrate mixture
Epoxidized linseed oil/Propyl citrate

| | | | |
|---|---|---|---|
| Day 0 | Day 12 | Day 22 | Cured @ |

| | | |
|---|---|---|
| Epoxy/Acid | Cured at room temperature | 140° C., |

| (Ep/Ac) groups | Tg0 | Tg12 | Tg22 | 1 hr |
|---|---|---|---|---|
| molar ratio | (° C.) | (° C.) | (° C.) | Tg (° C.) |
| 1.91 | 41.3 | 40.0 | 48.7 | |
| 1.79 | 39.8 | 44.6 | 42.0 | |
| 1.69 | 42.3 | 46.3 | 49.0 | 52.9 |
| 1.59 | 36.2 | 43.0 | 35.0 | |

When the epoxidized linseed oil replaced with the epoxidized hempseed oil, the optimum epoxy to carboxylic acid groups ratio was decreased from 1.7 to 1.6 (Table 14). This small difference could be due to their unique reactivities. Since the epoxidized linseed oil curing rate is faster than epoxidized hempseed oil, the mixture of epoxidized linseed oil and propyl citrate form a crosslinked network faster, which does not allow all of the epoxy groups to become part of the network, and this could the reason that the optimum Ep/Ac molar ratio is greater than 1 requiring higher amount of epoxy groups than equimolar amount.

TABLE 14
Optimum Ep/Ac groups molar ratio for the
epoxidized hempseed oil and propyl citrate mixture
Epoxidized hempseed oil/Propyl citrate

| | | | | Cured @ |
|---|---|---|---|---|
| Epoxy/Acid | Day 0 | Day 12 | Day 22 | 140° C., |
| (Ep/Ac) groups | Cured at room temperature | | | 1 hr |
| molar ratio | Tg0 (° C.) | Tg12 (° C.) | Tg22 (° C.) | Tg (° C.) |
| 1.67 | 20.4 | 27.9 | 28.4 | |
| 1.57 | 21.8 | 29.2 | 31.3 | 29.2 |
| 1.47 | 21.6 | 24.6 | 24.5 | |

### Example 9

### Ethyl Citrate Mixtures

The optimum epoxy to carboxylic acid groups ratio of 1.7 was determined for epoxidized linseed oil with the ethyl citrate (Table 15). Interestingly, the sample cured at 25° C. over time appears to be maximally cured in 12 days as the 22 day sample does not show any further increase in Tg. However, the bioresin prepared at elevated temperature has about 5° C. higher Tg than the sample cured at 25° C.

TABLE 15
Optimum Ep/Ac groups molar ratio for the
epoxidized linseed oil and ethyl citrate mixture
Epoxidized linseed oil/Ethyl citrate

| | | | | Cured @ |
|---|---|---|---|---|
| Epoxy/Acid | | | | 140° C., |
| (Ep/Ac) groups | Day 0 | Day 12 | Day 22 | 1 hr |
| molar ratio | Tg0 (° C.) | Tg12 (° C.) | Tg22 (° C.) | Tg (° C.) |
| 1.86 | 35.4 | 43.8 | 44.3 | |
| 1.74 | 38.7 | 40.3 | 40.1 | 45.1 |
| 1.63 | 33.4 | 39.4 | 37.1 | |

The optimum epoxy to carboxylic acid groups ratio was decreased from 1.7 to 1.4 when the epoxidized linseed oil replaced with the epoxidized hempseed oil with the ethyl citrate curing agent (Table 16). Additionally, a similar 25° C. curing rate phenomenon was observed so that most of the curing occurred within 12 days and there was no further significant change in Tg, when bioresin cured for longer. For this epoxidized hempseed oil/ethyl citrate case it was also observed that there was no significant difference in Tg between bioresins prepared at elevated temperature vs. cured at 25° C. Hence, it can be concluded that such bioresin mixture can maximally cured at 25° C. within 12 days.

TABLE 16
Optimum Ep/Ac groups molar ratio for the
epoxidized hempseed oil and ethyl citrate mixture
Epoxidized hempseed oil/Ethyl citrate

| Day 0 | | Day 12 | Day 22 | |
|---|---|---|---|---|
| Epoxy/Acid | Tg0 | Tg12 | Tg22 | Tg (Cured in oven |
| groups ratio | (° C.) | (° C.) | (° C.) | @ 140° C., 1 hrs.) |
| 1.63 | 20.2 | 22.2 | 24.8 | |
| 1.52 | 23.0 | 26.2 | 25.1 | |
| 1.42 | 23.8 | 23.4 | 23.0 | 24.1 |
| 1.34 | 20.2 | 23.1 | 26.4 | |

### Example 10

### Flexural Properties of Biocomposites

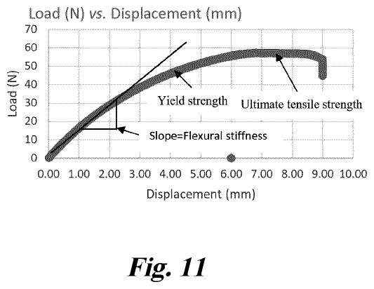

Five specimens for each test were prepared according to ASTM D7264/D7264M-15 and used for repeatability. A representative stress (N) versus deflection (mm) graph is shown in FIG. 11.

The initial linear portion of the curve was used to calculate the flexural stiffness of the specimens. The maximum load was considered as the ultimate tensile strength. These points are also shown in FIG. 11. The averages of the flexural stiffness and the ultimate tensile strength for the specimens tested for each case is presented in Tables 17-19.

The ultimate tensile strength and flexural stiffness were higher for the biocomposites that were prepared from epoxidized linseed oil compared to the biocomposites prepared from the epoxidized hempseed oil (Table 17). These higher mechanical properties of epoxidized linseed oil based biocomposites are due to higher % OOC of the epoxidized linseed oil.

TABLE 17
Flexural stiffness of biocomposite prepared by using epoxidized
hempseed-linseed oils with propyl citrate curing agent

Curing agent
Propyl citrate

| Epoxidized oil | Hempseed | Linseed |
|---|---|---|
| Ultimate tensile strength | 33.4 ± 2.8 | 58.2 ± 2.1 |
| (N/mm²) | | |
| Flexural stiffness (N/mm²) | 7.6 ± 0.9 | 10.6 ± 1.8 |
| Resin content (%) | 48.9 | 50.6 |
| Density (g/cm³) | 1.0 | 1.1 |

Similar superior mechanical properties were recorded for the biocomposites that were prepared by using ethyl and butyl citrates with epoxidized linseed oil vs hempseed oil. The biocomposites prepared from ethyl and propyl citrates were found to have better mechanical performance than the biocomposite prepared from butyl citrate. This could be due to the 14% of the plasticizing compound, dibutyl citrate, present in butyl citrate curing agent, and there were no such compounds present in other two CA-alkyl esters. This data is also in agreement with the Tg data from the DSC (Tables 14-16). Despite having slightly different composition between ethyl and propyl citrates (Table 10), the ultimate tensile strength and flexural stiffness differences between these two biocomposites were within standard deviation. Such negligible difference could be because of a small carboxylic acid functionality difference between ethyl citrate (2.2) and propyl citrate (2.3).

TABLE 18
Flexural stiffness of biocomposite prepared by using epoxidized
hempseed/linseed oils with ethyl citrate curing agent

Curing agent
Ethyl citrate

| Epoxidized oil | Hempseed | Linseed |
|---|---|---|
| Ultimate tensile strength | 33.6 ± 1.6 | 60.9 ± 3.9 |
| (N/mm²) | | |
| Flexural stiffness (N/mm²) | 7.6 ± 1.1 | 11.4 ± 1.5 |
| Resin content (%) | 42.4 | 49.0 |
| Density (g/cm³) | 1.0 | 1.1 |

In spite of differences in the resin amounts, 6.4% for hempseed and 1.6% for linseed, the mechanical stiffnesses of the biocomposites were negligible (Tables 17-18). Similarly, there were not significant density differences between biocomposites.

The ultimate tensile strength of the biocomposites prepared from butyl citrate was the lowest among the 3 types of curing agents because of its composition (Table 19), which includes dibutyl citrate resulting in the lowest functionality. The density of biocomposites prepared from butyl citrate and hempseed epoxides was also the lowest at 0.98 g/cm³ result in low crosslinking density polymer network, although this could also be possibly a result of sample preparation.

TABLE 19
Flexural stiffness of biocomposite prepared by using epoxidized
hempseed/linseed oils with butyl citrate curing agent

Curing agent
Butyl citrate

| Epoxidized oil | Hempseed | Linseed |
|---|---|---|
| Ultimate tensile strength | 15.9 ± 0.8 | 37.67 ± 5.7 |
| (N/mm²) | | |
| Flexural stiffness (N/mm²) | 5.4 ± 1.1 | 6.8 ± 1.3 |
| Resin content (%) | 45.4 | 44.9 |
| Density (g/cm³) | 1.0 | 1.2 |

### Example 11

Fully biobased bioresins and biocomposites were prepared without the use of solvent. Once these objective, fully biobased and solvent-free process, had been achieved then systematically viscoelastic properties of these bioresins and biocomposites were measured by dynamic mechanical analyzer (Table 20). Each bioresin and biocomposite sample was prepared in duplicate and their properties were measured to investigate the effect of the biobased curing agent on the properties of the final cured samples. Since the butyl citrate curing agent has substantial amount of dibutyl citrate which acts as a plasticizer it was of interest to see what effect this has on the cured sample compared to cured materials prepared using ethyl and propyl citrates.

TABLE 20
Glass transition temperatures (Tg) of bioresins and biocomposites

| | Tg (° C.) | | | | | |
|---|---|---|---|---|---|---|
| Curing agent | EC | | PC | | BC | |
| Epoxide | Hempseed | Linseed | Hempseed | Linseed | Hempseed | Linseed |
| Bioresins | 41.9 ± 1.1 | 56.0 ± 0.5 | 45.7 ± 0.2 | 60.8 ± 0.5 | 39.7 ± 0.2 | 54.7 ± 0.6 |
| Biocomposites | 27.1 ± 0.6 | 41.0 ± 0.3 | 30.4 ± 0.2 | 44.2 ± 2.0 | 23.8 ± 1.1 | 37.5 ± 1.4 |

The impact of three different biobased curing agents on the final properties of the bioresins and biocomposites is significant. The curing agents used in this study was a mixture of two or more compounds as depicted in Table 10. First, the effect of the curing agent's composition was evaluated. One common compound present in these three curing agents was citric acid, although its amount in each curing agent was different. For instance, citric acid in ethyl citrate was 17.2%, propyl citrate 29.2%, and butyl citrate 22.0%. Therefore, theoretically, the higher citric acid content curing agent have higher functionality so it could produce material with highest Tg. At the same time, it is better to consider the overall functionality of the curing agent (Table 10) to make the predictions about the material properties. Using this assumption, the bioresin and biocomposite prepared by using propyl citrate should have the highest Tg, which is what was found in this study, where the bioresin and biocomposite prepared from propyl citrate had a Tg of 60.8 and 44.2° C., respectively when epoxidized linseed oil used. Similarly, when the epoxidized hempseed oil was used, the highest Tg for both bioresin and biocomposite were observed with propyl citrate, at 45.7 and 30.4° C. respectively. Thus, even though there are only small differences in carboxylic acid functionalities between 3 curing agents, the Tg and storage modulus of bioresins and biocomposites have significant differences. Such differences in mechanical properties could be partly due to the plasticizing effects from the non-reactive compounds present in the curing agents. Tri-and dialkyl citrates have functionality of 0 and 1, respectively, they are considered as plasticizer and contribute to lowering the Tg and storage modulus of the material. Since the butyl citrate contains the highest amount of these di- and tributyl citrates, material prepared from this curing agent possess the weaker mechanical performance than the other two material samples.

Another possible justification for having the lower Tg and mechanical strengths of the materials prepared by using butyl citrate compared to ethyl- and propyl-citrates due to the difference is the carbon chain length. Butyl citrate contains 4 carbon compared to 2 and 3 for the ethyl- and propyl-citrates, respectively.

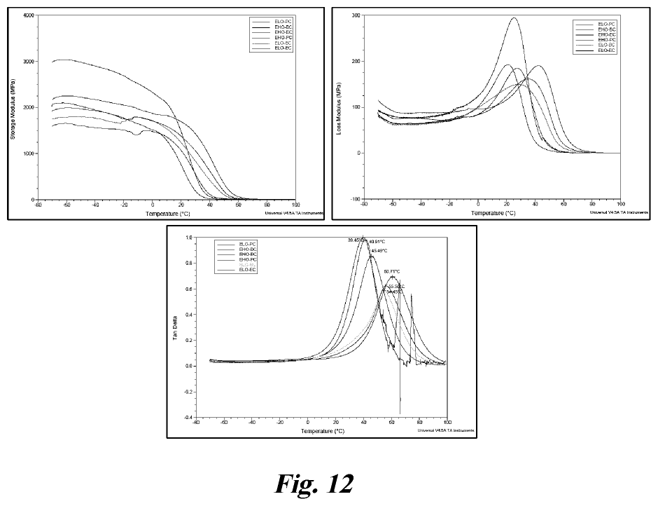

The viscoelastic behaviors of the bioresins are illustrated in FIG. 12 with their storage and loss modulus, and tan delta curves. The bioresin's storage modulus from 20 to 40° C. was highest (˜1800 MPa) for the epoxidized linseed oil with the ethyl and propyl citrates because of superior functionality as well as no plasticizing compound in both curing agents. In contrast, the storage modulus of the biocomposite (˜500 MPa) prepared from butyl citrate and hempseed oil epoxide was the lowest (FIG. 12). Similar effects were observed from the tan delta curves, and in addition the biocomposites prepared from linseed oil epoxide and ethyl and propyl citrates had the highest Tg (55.5 and 60.7° C.).

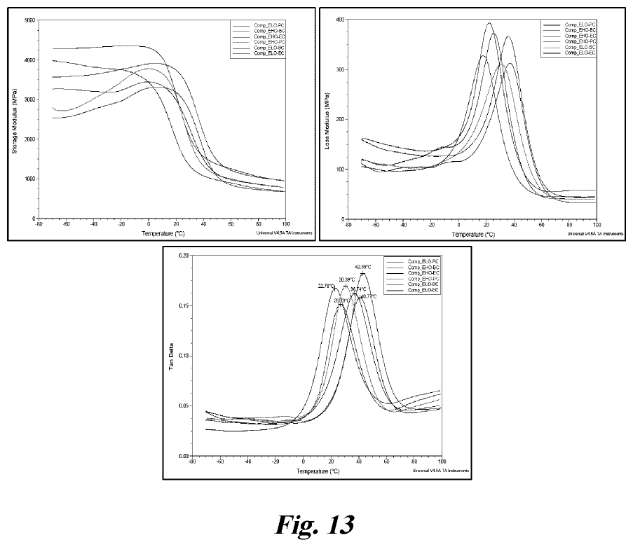

Similar viscoelastic behaviors were observed for the biocomposites such as the maximum storage modulus ˜2800 MPa was recorded between 20 to 30° C. for the linseed epoxide with the ethyl and propyl citrates (FIG. 13). Additionally, the viscoelastic properties and Tg values of bioresin and biocomposites were also in agreement.

Overall, these fully biobased resins possess properties that match with commercial resin products that are partially biobased. The Super Sap resin brand offers a range of epoxy resins and hardeners, and it is commercially produced by Entropy Resins, and all of their resins contain 21-30% biocontent by mass. The properties of five commercial epoxy resins produced by Entropy Resins were reported, and some of the properties are comparable with the fully biobased resins prepared in this work. Super Sap resin's cure cycle at 25° C. is from 3 days to 10 days with a post cure recommended, while fully biobased resins cure between 2 (tack-free state) to 15 (fully cured) days. The cured Super Sap resin's Tg (DSC) are between 63° C. and 115° C., while the fully biobased resin's (ELO/PC) Tg is 52° C. is achieved in this work.

A resole prepolymer was synthesized from cardanol (a phenolic by-product of cashew nut industry) and diglycidyl ether of bisphenol A (DGEBA) to prepare a thermoset resin, and such resin used to prepare biocomposites. These resin contains 40% biobased cardanol, and their Tg (DMA) was in a range of 42-56° C. while using silane compound as curing agent in presence of amine catalyst. In contrast, the Tg (DMA) in a range of 39 to 60° C. of fully biobased resins prepared from epoxidized plant oils and CA-alkyl esters curing agents. Additionally, the tensile strength of biocomposites prepared from 40% biobased phenolic resin was in a range of 9 to 20 MPa, and the biocomposites prepared from fully biobased resin possess tensile strength in a range of 15 to 60 MPa (Tables 17-19).

### Example 12

### Synthesis of Propenyl Citrate

Citric acid and ally! alcohol were used to prepare alkenyl citrate by adopting the reaction conditions used to prepare propyl citrate (Example 3). The reaction was performed at 110° C. under reflux conditions; the citric acid to ally! alcohol mole ratio was 1:2; and the reaction time was 2 hours.

The alkenyl citrate mixture was quantified by HPLC-ELSD and found that it contains about 46% unreacted citric acid and 54% mono alkenyl citrate without the formation of di- and tri-alkenyl citrates.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 63/075,940, filed Sep. 9, 2020, which application is hereby incorporated by reference in its entirety.

1. A method of preparing a curing agent, comprising the following steps:
a. providing citric acid and an alkyl or alkenyl alcohol in a suspension;
b. mixing and partially esterifying the suspension under reflux conditions to form a monoalkyl or monoalkenyl citrate curing agent substantially free of trialkyl or trialkenyl citrate;
wherein the alkyl alcohol is ethanol, n-propanol or n-butanol; and
wherein the alkenyl alcohol is allyl alcohol.

2. The method of claim 1, wherein the suspension upon mixing becomes a single phase in the absence of any added solvent.

3. The method of claim 1, wherein the curing agent is substantially free of dialkyl or dialkenyl citrate.

4. The method of claim 1, wherein the alkyl alcohol is ethanol.

5. The method of claim 1, wherein the alkyl alcohol is n-propanol.

6. The method of claim 1, wherein the alkyl alcohol is n-butanol.

7. The method of claim 1, wherein esterifying the citric acid occurs above the boiling point of the alcohol and below about 130° C.

8. The method of claim 1, wherein the ratio of citric acid to alcohol is between about 1:0.5 (mole/mole) to about 1:4 (mole/mole).

9. The method of claim 8, wherein the ratio of citric acid to alcohol is between about 1:0.5 (mole/mole) to about 1:2 (mole/mole).

10. The method of claim 1, wherein the monoalkyl citrate is present in about 60 wt % to about 85 wt % in presence with unreacted citric acid.

11. The method of claim 10, wherein the monoalky citrate is present in about 75 wt % to about 85 wt % in presence with unreacted citric acid.

12. A curing agent prepared according to the method of claim 1.

13. A method of preparing a thermoset polymer comprising reacting free carboxylic acids present in a monoalkyl citrate curing agent with epoxy monomers.

14. The method of claim 13, wherein preparing the thermoset polymer further comprises curing the epoxy monomers at room temperature.

15. The method of claim 13, wherein reacting the monoalkyl citrate curing agent with epoxy monomers occurs without solvent.

16. The method of claim 13, wherein the monoalkyl citrate curing agent is miscible with epoxy monomers at room temperature.

17. The method of claim 13, wherein the monoalkyl citrate curing agent and epoxy monomers remain a single phase mixture at room temperature.

18. The method of claim 13, wherein the epoxy monomers are derived from unsaturated triglycerides in plant oil.

19. The method of claim 18, wherein the plant oil is hemp oil.

20. A thermoset polymer prepared according to the method of claim 13.

21. (canceled)

22. (canceled)
